(19) United States
(12) Patent Application Publication
(10) Pub. No.: US 2022/0387376 A1
(43) Pub. Date: Dec. 8, 2022

(54) Compositions Comprising Cannabinoid Molecules that are Dissolved in Water-Miscible Solvents

(71) Applicant: Natural Extraction Systems, LLC, Boulder, CO (US)

(72) Inventors: C. Russell Thomas, Boulder, CO (US); Douglas G. Metcalf, Boulder, CO (US)

(21) Appl. No.: 17/769,938

(22) PCT Filed: Oct. 16, 2020
(86) PCT No.: PCT/US20/56046

§ 371 Date: Apr. 18, 2022

Related U.S. Application Data
(60) Provisional application No. 62/923,412, filed on Oct. 18, 2019.

(51) Int. Cl. A61K 31/352; A61K 9/08; A61K 47/10; A61K 31/05

(52) U.S. Cl. CPC A61K 31/352; A61K 9/08; A61K 47/10; A61K 31/05

(57) ABSTRACT

Various aspects of this patent document relate to cannabinoids that are dissolved in glycerine in the presence of the cosolvent ethanol.

### CROSS-REFERENCE TO RELATED APPLICATIONS

This patent application claims priority to U.S. Provisional Patent Application No. 62/923,412, filed Oct. 18, 2019, which is incorporated by reference in its entirety.

### BACKGROUND

Cannabinoids consumed through conventional routes such as by inhalation or oral ingestion are oxidized by cytochrome P450 in the lungs and liver, respectively. Cannabinoid formulations that minimize or avoid cytochrome P450 mediated oxidation are desirable.

### SUMMARY

Various aspects of this patent document relate to cannabinoid molecules that are dissolved in water-miscible solvents. The cannabinoid molecules rapidly partition out of the water-miscible solvents upon administration to an organism, for example, and adhere to the epithelium where they absorb into the blood (during oral administration) or the skin and underlying tissue (during topical administration). Absorption through the epithelium of the mouth, throat, or skin avoids first-pass metabolism, which minimizes cytochrome P450 mediated oxidation and improves pharmacokinetics.

### DETAILED DESCRIPTION

Various aspects of this patent document relate to a composition, a cannabinoid, ethanol, and a solvent, wherein: the composition comprises a liquid phase that comprises the cannabinoid, the ethanol, and the solvent; the cannabinoid is a molecule that lacks a net charge; the cannabinoid is a solute that is dissolved in the solvent; the ethanol is a cosolvent; the liquid phase comprises the solvent at a concentration of at least 50 percent by mass; the solvent is an alcohol that has the formula CxHyOz; the alcohol consists of atoms that are connected by single bonds (and that are not connected by any double bonds or triple bonds); x is an integer that is greater than 2; z is an integer that is greater than 1 and no greater than x; and either (i) the alcohol is linear, and y=2x+2 or (ii) the alcohol contains a monocycle, and y=2x.

In all embodiments, the cannabinoid is neither a carboxylic acid nor a carboxylate. For example, in all embodiments, the cannabinoid is neither cannabidiolic acid nor tetrahydrocannabinolic acid.

“Comprising” and “comprise(s)” refer to open-ended sets such that a composition that comprises a cannabinoid that is a molecule that lacks a net charge can also comprise a cannabinoid that is an anion that has a net negative charge. “Consists of” refers to a closed set.

“Glycerine” is synonymous with glycerin, glycerol, and propane-1,2,3-triol.

“Dissolved” refers to a solute that is solvated in a liquid phase by either (i) a solvent, (ii) a cosolvent, or (iii) both a solvent and a cosolvent; a chemical species that is present within a phase that is dispersed within a liquid phase, such as the dispersed phase of an emulsion, is not dissolved in the liquid phase; a chemical species that is non-covalently bound to any chemical species that is a solid in the absence of a solvent, such as a cyclodextrin, is not dissolved in a solvent.

In some embodiments, x is at least 2 and no greater than 7; and z is equal to either x or x minus 1. In some specific embodiments, the solvent is propylene glycol; propane-1,3-diol; glycerine, erythritol, threitol, arabitol, xylitol, ribitol, mannitol, sorbitol, galactitol, fucitol, iditol, inositol, or volemitol. In some very specific embodiments, the solvent is glycerine.

In some embodiments, the solvent is polyethylene glycol.

Various aspects of this patent document relate to a composition, comprising a cannabinoid, ethanol, and glycerine, wherein: the composition is a liquid; the cannabinoid is a molecule that lacks a net charge; the cannabinoid is a solute; the glycerine is a solvent; the ethanol is a cosolvent; and the cannabinoid is dissolved in the glycerine.

In some embodiments, the ethanol of a composition inhibits the cannabinoid from forming either a precipitate, lipid phase, or flocculant in the solvent.

In some embodiments, the composition comprises glycerine at a concentration of at least 50 percent by mass. In some specific embodiments, the composition comprises the glycerine at a concentration of at least 58.5 percent and no greater than 86.4 percent by mass; the ethanol at a concentration of at least 13.0 percent and no greater than 38.5 percent by mass; and the cannabinoid at a concentration of at least 0.01 percent and no greater than 4.9 percent by mass.

In some embodiments, the composition comprises the ethanol and the cannabinoid at a molecular ratio of at least 21:1.

In some embodiments, the cannabinoid has a solubility in water, and the composition comprises the cannabinoid at a concentration that is greater than the solubility of the cannabinoid in water. In some specific embodiments, the composition comprises the cannabinoid at a concentration that is at least two times greater than the solubility of the cannabinoid in water. In some very specific embodiments, the composition comprises the cannabinoid at a concentration that is at least five times greater than the solubility of the cannabinoid in water.

In some embodiments, the cannabinoid has a solubility in glycerine, and the composition comprises the cannabinoid at a concentration that is greater than the solubility of the cannabinoid in glycerine. In some specific embodiments, the composition comprises the cannabinoid at a concentration that is at least two times greater than the solubility of the cannabinoid in glycerine. In some very specific embodiments, the composition comprises the cannabinoid at a concentration that is at least five times greater than the solubility of the cannabinoid in glycerine.

In some embodiments, the composition comprises the cannabinoid at a concentration of at least 1 gram per liter. In some specific embodiments, the composition comprises the cannabinoid at a concentration of at least 5 grams per liter. In some very specific embodiments, the composition comprises the cannabinoid at a concentration of at least 10 grams per liter.

In some embodiments, the composition comprises a metal cation. In some specific embodiments, the metal cation is sodium cation (“Na+”) or potassium cation (“K+”). In some embodiments, the composition comprises a concentration of the metal cation and a concentration of the cannabinoid, and the concentration of the metal cation in the composition is greater than the concentration of the cannabinoid in the composition. In some specific embodiments, the concentration of the metal cation in the composition is at least two times greater than the concentration of the cannabinoid in the composition. In some very specific embodiments, the concentration of the metal cation in the composition is at least five times greater than the concentration of the cannabinoid in the composition.

In some embodiments, the composition comprises water.

In some embodiments, the composition comprises an alkaline pH. The term “alkaline pH,” as used in this patent document, refers to a composition that comprises a solvent that is an alcohol (for example glycerine), one or more conjugate bases of the solvent (for example, 2,3-dihydroxy-propane-1-oxide or 1,3-dihydroxy-propane-2-oxide), and optionally one or more conjugate acids of the solvent (for example, 2,3-dihydroxy-propane-1-oxonium or 1,3-dihydroxy-propane-2-oxonium), wherein the combined molar concentration of the one or more conjugate bases of the solvent in the composition is greater than the combined molar concentration of any conjugate acids of the solvent in the composition.

In some embodiments, the composition comprises hydroxide.

In some embodiments, the composition comprises ethoxide.

In some embodiments, the composition comprises 2,3-dihydroxy-propane-1-oxide or 1,3-dihydroxy-propane-2-oxide.

In some embodiments, the cannabinoid has a general formula I, II, III, or IV.

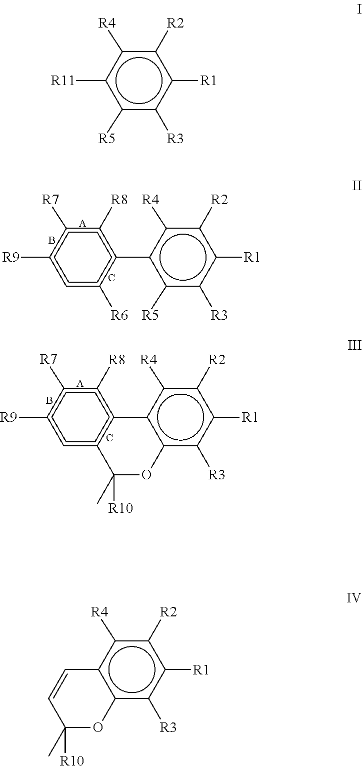

In some embodiments, R1 is selected from H; a straight or branched C1-C12 alkyl that is optionally substituted by at least one of hydroxy, a halogen, phenyl, and a cycloalkyl; and a straight or branched C2-C12 alkenyl that is optionally substituted by at least one of hydroxy, a halogen, phenyl, and a cycloalkyl. “Straight or branched C1-C12 alkyl” refers to a straight or branched hydrocarbon chain having 1, 2, 3, 4, 5, 6, 7, 8, 9, 10, 11, or 12 carbon atoms, wherein all carbon-carbon bonds in the hydrocarbon chain are single bonds. “Substituted by at least one of hydroxy, a halogen, phenyl, and a cycloalkyl” refers to the substitution of at least one hydrogen atom of a hydrocarbon chain with either hydroxy, a halogen, phenyl, or a cycloalkyl. “Halogen” refers to F, Cl, Br, and I. “Cycloalkyl” refers to cyclopropyl, cyclobutyl, cyclopentyl, cyclohexyl, cycloheptyl, and adamantyl. When a hydrocarbon chain is substituted by a cycloalkyl, then either (i) a single hydrogen atom of the hydrocarbon chain is substituted with the cycloalkyl such that the cycloalkyl does not include any carbon atom of the hydrocarbon chain, or (ii) two hydrogen atoms of the hydrocarbon chain are substituted with the cycloalkyl such that the cycloalkyl comprises one or more carbon atoms of the hydrocarbon chain. “Straight or branched C2-C12 alkenyl” refers to a straight or branched hydrocarbon chain having 2, 3, 4, 5, 6, 7, 8, 9, 10, 11, or 12 carbon atoms, wherein at least one carbon-carbon bond in the hydrocarbon chain is a double bond and no carbon-carbon bond in the hydrocarbon chain is a triple bond.

In some embodiments, R2, R3, R8, and R9 are each independently selected from H and a halogen. In some specific embodiments, each of R2, R3, R8, and R9 are H. In some specific embodiments, R2 is F; and each of R3, R8, and R9 are H. In some specific embodiments, R3 is F; and each of R2, R8, and R9 are H. In some specific embodiments, R8 is F; and each of R2, R3, and R9 are H. In some specific embodiments, R9 is F; and each of R2, R3, and R8 are H.

In some embodiments, R4 and R5 are each independently selected from H, hydroxy, methoxy, ethoxy, 2-propoxy, and —OC(═O)R12.

In some embodiments, R6 is selected from H; a halogen; a straight of branched C1-C6 alkyl that is optionally substituted by at least one of hydroxy and a halogen; a straight of branched C2-C₆ alkenyl that is optionally substituted by at least one of hydroxy and a halogen; and a straight of branched C2-C₆ alkynyl that is optionally substituted by at least one of hydroxy and a halogen. “Straight or branched C1-C6 alkyl” refers to a straight or branched hydrocarbon chain having 1, 2, 3, 4, 5, or 6 carbon atoms, wherein all carbon-carbon bonds in the hydrocarbon chain are single bonds. “Straight or branched C2-C₆ alkenyl” refers to a straight or branched hydrocarbon chain having 2, 3, 4, 5, or 6 carbon atoms, wherein at least one carbon-carbon bond in the hydrocarbon chain is a double bond and no carbon-carbon bond in the hydrocarbon chain is a triple bond. “Straight or branched C2-C₆ alkynyl” refers to a straight or branched hydrocarbon chain having 2, 3, 4, 5, or 6 carbon atoms, wherein at least one carbon-carbon bond in the hydrocarbon chain is a triple bond. “Substituted by at least one of hydroxy and a halogen” refers to the substitution of at least one hydrogen atom of a hydrocarbon chain with either hydroxy or a halogen.

In some embodiments, R7 is selected from H; a halogen; hydroxy; oxo; methylidene; a straight or branched C1-C6 alkyl that is optionally substituted by at least one of hydroxy and a halogen; a straight or branched C2-C₆ alkenyl that is optionally substituted by at least one of hydroxy and a halogen; a straight or branched C2-C₆ alkynyl that is optionally substituted by at least one of hydroxy and a halogen; and —C(═O)R13.

In some embodiments, R10 is selected from H; a halogen; hydroxy; a straight of branched C1-C6 alkyl that is optionally substituted by at least one of hydroxy and a halogen; a straight of branched C2-C₆ alkenyl that is optionally substituted by at least one of hydroxy and a halogen; and a straight of branched C2-C₆ alkynyl that is optionally substituted by at least one of hydroxy and a halogen.

In some embodiments, R11 is selected from a straight or branched C5-C12 alkyl that is optionally substituted by at least one of hydroxy, a halogen, phenyl, and a cycloalkyl; a straight or branched C5-C12 alkenyl that is optionally substituted by at least one of hydroxy, a halogen, phenyl, and a cycloalkyl; and a straight or branched C5-C12 alkynyl that is optionally substituted by at least one of hydroxy, a halogen, phenyl, and a cycloalkyl. “Straight or branched C5-C12 alkyl” refers to a straight or branched hydrocarbon chain having 5, 6, 7, 8, 9, 10, 11, or 12 carbon atoms, wherein all carbon-carbon bonds in the hydrocarbon chain are single bonds. “Straight or branched C5-C12 alkenyl” refers to a straight or branched hydrocarbon chain having 5, 6, 7, 8, 9, 10, 11, or 12 carbon atoms, wherein at least one carbon-carbon bond in the hydrocarbon chain is a double bond and no carbon-carbon bond in the hydrocarbon chain is a triple bond. “Straight or branched C5-C12 alkynyl” refers to a straight or branched hydrocarbon chain having 5, 6, 7, 8, 9, 10, 11, or 12 carbon atoms, wherein at least one carbon-carbon bond in the hydrocarbon chain is a triple bond.

In some embodiments, R12 is selected from a straight or branched C1-C6 alkyl.

In some embodiments, R13 is selected from H; a straight or branched C1-C6 alkyl; and a straight or branched C1-C6 alkoxy. “Straight or branched C1-C6 alkoxy” refers to —OR14, wherein R14 is a straight or branched C1-C6 alkyl.

In some embodiments, the dotted lines in general formulas II and III depict the bonding pattern of either cyclohexane, phenyl, or a cyclohexene that comprises exactly 1 double bond, which occurs at either A, B, or C.

In some embodiments, R1 is methyl; ethyl; propyl; butyl; pentyl; hexyl; heptyl; octyl; nonyl; decyl; prop-2-yl; but-2-yl; pent-2-yl; hex-2-yl; hept-2-yl; octan-2-yl; nonan-2-yl; decan-2-yl; 2-methylpropyl; 2-methylbutyl; 2-methylpentyl; 2-methylhexyl; 2-methylheptyl; 2-methyloctyl; 2-methylnonyl; 2-methyldecyl; 2-methylprop-2-yl; 2-methylbut-2-yl; 2-methylpent-2-yl; 2-methylhex-2-yl; 2-methylhept-2-yl; 2-methyloctan-2-yl; 2-methylnonan-2-yl; 2-methyldecan-2-yl; 3-methylbut-2-yl; 3-methylpent-2-yl; 3-methylhex-2-yl; 3-methylhept-2-yl; 3-methyloctan-2-yl; 3-methylnonan-2-yl; 3-methyldecan-2-yl; 2,3-dimethylbut-2-yl; 2,3-dimethylpent-2-yl; 2,3-dimethylhex-2-yl; 2,3-dimethylhept-2-yl; 2,3-dimethyloctan-2-yl; 2,3-dimethylnonan-2-yl; 2,3-dimethyldecan-2-yl; cyclopropyl; 1-methylcyclopropyl; 1-ethylcyclopropyl; 1-propylcyclopropyl; 1-butylcyclopropyl; 1-pentylcyclopropyl; 1-hexylcyclopropyl; 1-heptylcyclopropyl; 1-octylcyclopropyl; 1-nonylcyclopropyl; cyclobutyl; 1-methylcyclobutyl; 1-ethylcyclobutyl; 1-propylcyclobutyl; 1-butylcyclobutyl; 1-pentylcyclobutyl; 1-hexylcyclobutyl; 1-heptylcyclobutyl; 1-octylcyclobutyl; cyclopentyl; 1-methylcyclopentyl; 1-ethylcyclopentyl; 1-propylcyclopentyl; 1-butylcyclopentyl; 1-pentylcyclopentyl; 1-hexylcyclopentyl; 1-heptylcyclopentyl; cyclohexyl; 1-methylcyclohexyl; 1-ethylcyclohexyl; 1-propylcyclohexyl; 1-butylcyclohexyl; 1-pentylcyclohexyl; 1-hexylcyclohexyl; ethenyl; prop-1-enyl; but-1-enyl; pent-1-enyl; hex-1-enyl; hept-1-enyl; octan-1-enyl; nonan-1-enyl; decan-1-enyl; ethynyl; prop-1-ynyl; but-1-ynyl; pent-1-ynyl; hex-1-ynyl; hept-1-ynyl; octan-1-ynyl; nonan-1-ynyl; decan-1-ynyl; 2-phenylethyl; or adamantyl. In some specific embodiments, R1 is propyl or pentyl.

In some embodiments, R2 is H.

In some embodiments, R3 is H.

In some embodiments, R4 is hydroxy.

In some embodiments, R5 is hydroxy.

In some embodiments, R6 is H; 2-propyl; propen-2-yl; 3-hydroxypropyl; 3-hydroxypropen-2-yl; 4-hydroxy-1-methylbutyl; 4-hydroxy-1-methylbut-2-enyl; 4-hydroxy-1-methylbut-2-ynyl; 1-fluoroethenyl; or 3-fluoropropen-2-yl. In some specific embodiments, R6 is propen-2-yl.

In some embodiments, R7 is methyl, hydroxymethyl, fluoromethyl, or oxo. In some specific embodiments, R7 is methyl.

In some embodiments, R8 is H.

In some embodiments, R9 is H.

In some embodiments, R10 is H; methyl; 4-methylpent-3-enyl; hydroxymethyl; 3-hydroxypropyl; 3-hydroxyprop-1-enyl; 3-hydroxyprop-1-ynyl; fluoro; or fluoromethyl. In some specific embodiments, R10 is methyl.

In some embodiments, R11 is 3-methylbut-2-enyl or 3,7-dimethylocta-2,6-dienyl. In some specific embodiments, R11 is 3,7-dimethylocta-2,6-dienyl.

In some embodiments, R12 is methyl.

In some embodiments, R13 is H, methoxy, ethoxy, or 2-propoxy.

In some embodiments, the cannabinoid has a general formula II or III; the dotted lines in general formulas II and III depict the bonding pattern of a cyclohexene; and the cyclohexene comprises exactly 1 double bond, which occurs at either A, B, or C. In some specific embodiments, the cannabinoid has a general formula II or III; the dotted lines in general formulas II and III depict the bonding pattern of a cyclohexene; and the cyclohexene comprises exactly 1 double bond, which occurs at A. In some specific embodiments, the cannabinoid has a general formula II or III; the dotted lines in general formulas II and III depict the bonding pattern of a cyclohexene; and the cyclohexene comprises exactly 1 double bond, which occurs at B. In some specific embodiments, the cannabinoid has a general formula II or III; the dotted lines in general formulas II and III depict the bonding pattern of a cyclohexene; and the cyclohexene comprises exactly 1 double bond, which occurs at C.

In some embodiments, the cannabinoid has a general formula II or III; and the dotted lines in general formulas II and III depict the bonding pattern of phenyl.

In some embodiments, the cannabinoid has a general formula II or III; and the dotted lines in general formulas II and III depict the bonding pattern of cyclohexyl.

In some embodiments, the cannabinoid is selected from cannabidiol (“CBD”); cannabidivarin (“CBDV”); cannabidiorcol (“CBD-C1”); cannabidiphorol (“CBD-C7”); tetrahydrocannabinol (“THC”); tetrahydrocannabivarin (“THCV”); tetrahydrocannabiorcol (“THC-C1”); tetrahydrocannabiphorol (“THC-C7”); cannabinol; cannabivarin; cannabigerol; cannabigerovarin; cannabichromene; cannabichromevarin; perrottetinene; delta8-tetrahydrocannabinol; delta8-tetrahydrocannabivarin; 11-hydroxy-tetrahydrocannabinol; 11-hydroxy-tetrahydrocannabivarin; cannabidiol-dimethylheptyl; cannabicyclohexanol; canbisol; nabilone; dexanabinol; parahexyl; dimethylheptylpyran; pirnabine; 11-fluoro-tetrahydrocannabinol; 11-fluoro-tetrahydrocannabivarin; 4′-fluorocannabidiol; HU-331; HU-336; and HU-345.

In some specific embodiments, the cannabinoid is cannabidiol.

In some specific embodiments, the cannabinoid is cannabidivarin.

In some specific embodiments, the cannabinoid is tetrahydrocannabinol.

In some specific embodiments, the cannabinoid is tetrahydrocannabivarin.

In some specific embodiments, the cannabinoid is cannabigerol.

In some specific embodiments, the cannabinoid is cannabigerovarin.

In some embodiments, the composition comprises the cannabinoid at a concentration of at least 10 grams per liter and no greater than 100 grams per liter.

In some embodiments, the composition comprises the cannabinoid at a concentration of at least 1 gram per liter and no greater than 10 grams per liter.

In some embodiments, the composition comprises the cannabinoid at a concentration of at least 100 milligrams per liter and no greater than 1 gram per liter.

In some embodiments, the composition comprises the cannabinoid at a concentration of at least 10 milligrams per liter and no greater than 100 milligrams per liter.

In some embodiments, the composition comprises the cannabinoid at a concentration of at least 1 milligram per liter and no greater than 10 milligrams per liter.

In some embodiments, the composition is formulated for oral administration to a human or an animal. In some embodiments, the composition is formulated for topical administration to a human or an animal.

Various aspects of this patent document relate to a medicament, comprising a composition described anywhere in this patent document.

Various aspects of this patent document relate to a method to administer a composition to a subject, comprising orally administering a composition described anywhere in this patent document to the subject, wherein the subject is a human or an animal.

Various aspects of this patent document relate to a method to administer a composition to a subject, comprising topically administering a composition described anywhere in this patent document to the subject, wherein the subject is a human or an animal.

In some embodiments, the animal is a rodent, lagomorph, feline, canine, porcine, ovine, lama, vicugna, bovine, equine, or primate. In some preferred embodiments, the subject is human.

EXEMPLIFICATION. Examples 1-11 set forth specific embodiments of this disclosure, and examples 1-11 do not limit the scope of the disclosure or any claim that matures from this patent document. Example 7-11 are adapted from International Application No. PCT/US20/48152, which is incorporated by reference for its disclosure of compositions that fall within the scope of this disclosure that comprise an alkaline pH.

### Example 1. 25 Milligrams of Cannabidiol Dissolved in Glycerine Displays a Robust Psychoactive Effect in Less than 5 Minutes

1.5 grams of cannabidiol was dissolved in 21 grams of 190 proof ethanol, and then 41.6 grams of glycerine was added to create a composition comprising approximately 2 percent cannabidiol, 31 percent ethanol, 2 percent water, and 65 percent glycerine by mass. The solution was transparent, which indicated that the cannabidiol completely dissolved in the glycerine. Three consenting adults each consumed approximately 1 milliliter of the composition containing approximately 25 milligrams of cannabidiol each and reported experiencing psychoactive effects including decreased anxiety and elevated mood within 5 minutes of consumption.

### Example 2. Cannabinoids are Insoluble in Glycerine, and Cannabinoids are not Necessarily Soluble in Mixtures of Glycerine and Ethanol

2 grams of cannabidiol was dissolved in 15 grams of 190 proof ethanol, and then 45 grams of glycerine was added to create a composition comprising 3 percent cannabidiol, 23 percent ethanol, 1 percent water, and 73 percent glycerine by mass. The solution was cloudy, which indicated that the cannabidiol did not completely dissolve in the glycerine. The solution was heated at 60 degrees Celsius in an attempt to dissolve the cannabidiol, but the cannabidiol melted and floated to the top of the mixture without dissolving.

### Example 3. Different Ratios of Cannabinoids, Glycerine, and Ethanol Result in Variable Solubility of the Cannabinoids in the Glycerine

600 milligrams of cannabidiol was dissolved in 17 grams of 190 proof ethanol, and then 55 grams of glycerine was added to create a composition comprising 0.8 percent cannabidiol, 22 percent ethanol, 1 percent water, and 76 percent glycerine by mass. The solution was transparent, which indicated that the cannabidiol completely dissolved in the glycerine.

### Example 4. The Results Obtained from Examples 1-3 are Summarized in Table 1 Below

TABLE 1
Percent by mass and solubility of CBD of the compositions described in Examples 1-3

| Sample | Cannabidiol | Ethanol | Water | Glycerine | Solubility |
|---|---|---|---|---|---|
| 1 | 2% | 31% | 2% | 65% | Dissolved |
| 2 | 3% | 23% | 1% | 73% | Insoluble |
| 3 | 0.8% | 22% | 1% | 76% | Dissolved |

### Example 5. Formulation of Cannabidiol in Glycerine Under Alkaline Conditions

Cannabidiol is dissolved in 0.5 molar potassium hydroxide in 190 proof ethanol, which is then combined with glycerine to produce a formulation comprising approximately 25 percent ethanol by volume and 25 grams cannabinoid per liter.

### Example 6. The Cannabidiol Formulation Displays Efficacy at Arresting Active Seizures in Canine

The FDA- and EMA-approved cannabidiol pharmaceutical EPIDIOLEX® is administered at 5-20 mg/kg/day to treat epilepsy, but EPIDIOLEX® is not known to arrest active seizures. A single oral dose of 0.5-1.0 mg/kg cannabinoid in a formulation prepared according to Example 5 both consistently and instantaneously arrested active seizures in an epileptic Entlebucher Mountain Dog resulting in reduced seizure duration and severity. Cytochrome P450 metabolizes a significant portion of EPIDIOLEX®. The rapid onset of the glycerine formulation suggests that at least a portion avoided first-pass metabolism.

### Example 7. Formulation of Cannabigerol Dissolved in Glycerine

Cannabigerol is dissolved in 0.5 molar potassium hydroxide in 190 proof ethanol, which is then combined with glycerine to produce a formulation comprising approximately 25 percent ethanol by volume and 25 grams cannabinoid per liter.

### Example 8. The Cannabigerol Formulation Displays Efficacy Against Insomnia in Humans

An adult human who presented with chronic insomnia consumed the cannabigerol formulation prepared according to Example 7 such that the individual ingested approximately 25 milligrams of cannabinoids. The individual reported a sedative effect that was effective at treating the insomnia with an onset time of less than five minutes. The rapid onset of the cannabigerol formulation suggests that at least a portion avoided first-pass metabolism.

### Example 9. Formulation of the Tetrahydrocannabinol Dissolved in Glycerine

Tetrahydrocannabinol is dissolved in 0.5 molar potassium hydroxide in 190 proof ethanol, which is then combined with glycerine to produce a formulation comprising approximately 25 percent ethanol by volume and 1 gram cannabinoid per liter.

### Example 10. The Tetrahydrocannabinol Formulation Displays Efficacy Against Insomnia in Humans

The psychoactive effects of tetrahydrocannabinol largely stem from its cytochrome P450 oxidation product 11-hydroxy-tetrahydrocannabinol. A standard oral dose of recreational tetrahydrocannabinol is 10 milligrams. An adult human orally consumed 10 milligrams of cannabinoids in a formulation prepared according to Example 9. The individual reported no marked psychoactive effect, which suggests that the cannabinoids may have avoided conversion into 11-hydroxy-tetrahydrocannabinol by avoiding first-pass metabolism.

### Example 11. Formulation of Cannabidivarin, Cannabichromene, and Cannabinol Dissolved in Glycerine

Distilled industrial hemp extract containing 79 percent cannabidiol, 4 percent cannabichromene, 1.6 percent tetrahydrocannabinol, 0.5 percent cannabidivarin, and 0.4 percent cannabinol was dissolved in 0.5 molar potassium hydroxide in 190 proof ethanol, which was then combined with glycerine to produce a formulation comprising approximately 25 percent ethanol by volume and approximately 27 milligrams cannabinoids per liter. The formulation was administered to humans as a tincture, and it displayed analgesic and anxiolytic effects.

What is claimed is:

1. A composition, comprising a cannabinoid, ethanol, and a solvent, wherein:
the composition comprises a liquid phase that comprises the cannabinoid, the ethanol, and the solvent;
the cannabinoid is a molecule that lacks a net charge;
the cannabinoid is a solute that is dissolved in the solvent;
the ethanol is a cosolvent;
the liquid phase comprises the solvent at a concentration of at least 50 percent by mass; and
the solvent is an alcohol that has the formula CxHyOz; the alcohol consists of atoms that are connected by single bonds; x is an integer that is greater than 2; z is an integer that is greater than 1 and no greater than x; and either (i) the alcohol is linear, and y=2x+2 or (ii) the alcohol contains a monocycle, and y=2x.

2. The composition of claim 1, wherein the solvent is glycerine or propylene glycol.

3. A composition, comprising a cannabinoid, ethanol, and glycerine, wherein:
the composition is a liquid;
the cannabinoid is a molecule that lacks a net charge;
the cannabinoid is a solute;
the glycerine is a solvent;
the ethanol is a cosolvent; and
the cannabinoid is dissolved in the glycerine.

4. The composition of claim 3, wherein the ethanol inhibits the cannabinoid from forming either a precipitate, lipid phase, or flocculant in the glycerine.

5. The composition of claim 3 or 4, comprising the glycerine at a concentration of at least 50 percent by mass.

6. The composition of any one of claims 3-5, comprising:
the glycerine at a concentration of at least 58.5 percent and no greater than 86.4 percent by mass;
the ethanol at a concentration of at least 13.0 percent and no greater than 38.5 percent by mass; and
the cannabinoid at a concentration of at least 0.01 percent and no greater than 4.9 percent by mass.

7. The composition of any one of claims 1-6, comprising the ethanol and the cannabinoid at a molecular ratio of at least 21:1.

8. The composition of any one of claims 1-7, wherein the cannabinoid has a solubility in water, and the composition comprises the cannabinoid at a concentration that is greater than the solubility of the cannabinoid in water.

9. The composition of any one of claims 1-8, wherein the cannabinoid has a solubility in glycerine, and the composition comprises the cannabinoid at a concentration that is greater than the solubility of the cannabinoid in glycerine.

10. The composition of any one of claims 1-9, comprising the cannabinoid at a concentration of at least 1 gram per liter.

11. The composition of any one of claims 1-10, comprising a metal cation.

12. The composition of claim 11, wherein the metal cation is sodium cation (“Na+”) or potassium cation (“K+”).

13. The composition of claim 11 or 12, wherein the composition comprises a concentration of the metal cation and a concentration of the cannabinoid, and the concentration of the metal cation in the composition is greater than the concentration of the cannabinoid in the composition.

14. The composition of claim 13, wherein the concentration of the metal cation in the composition is at least 2 times greater than the concentration of the cannabinoid in the composition.

15. The composition of any one of claims 1-14, comprising water.

16. The composition of any one of claims 1-15, comprising an alkaline pH.

17. The composition of any one of claims 1-16, comprising hydroxide.

18. The composition of any one of claims 1-17, comprising ethoxide.

19. The composition of any one of claims 1-18, comprising 2,3-dihydroxy-propane-1-oxide or 1,3-dihydroxy-propane-2-oxide.

20. The composition of any one of claims 1-19, wherein the cannabinoid has a general formula I, II, III, or IV 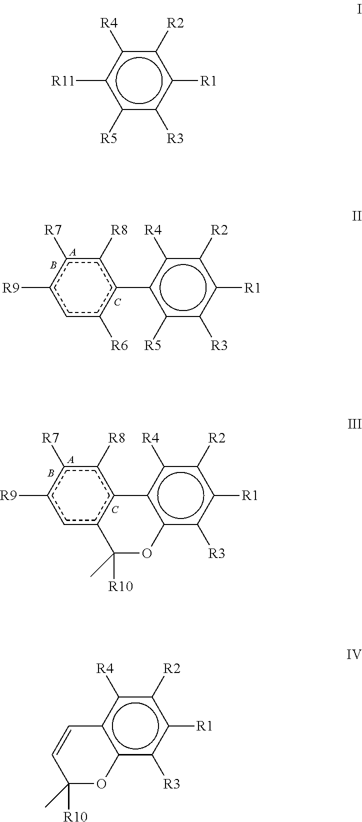
wherein:
R1 is selected from H; a straight or branched C1-C12 alkyl that is optionally substituted by at least one of hydroxy, a halogen, phenyl, and a cycloalkyl; and a straight or branched C2-C12 alkenyl that is optionally substituted by at least one of hydroxy, a halogen, phenyl, and a cycloalkyl;
R2, R3, R8, and R9 are each independently selected from H and a halogen;
R4 and R5 are each independently selected from H, hydroxy, methoxy, ethoxy, 2-propoxy, and —OC(═O)R12;
R6 is selected from H; a halogen; a straight of branched C1-C6 alkyl that is optionally substituted by at least one of hydroxy and a halogen; a straight of branched C2-C₆ alkenyl that is optionally substituted by at least one of hydroxy and a halogen; and a straight of branched C2-C₆ alkynyl that is optionally substituted by at least one of hydroxy and a halogen;
R7 is selected from H; a halogen; hydroxy; oxo; methylidene; a straight or branched C1-C6 alkyl that is optionally substituted by at least one of hydroxy and a halogen; a straight or branched C2-C6 alkenyl that is optionally substituted by at least one of hydroxy and a halogen; a straight or branched C2-C₆ alkynyl that is optionally substituted by at least one of hydroxy and a halogen; and —C(═O)R13;
R10 is selected from H; a halogen; hydroxy; a straight of branched C1-C6 alkyl that is optionally substituted by at least one of hydroxy and a halogen; a straight of branched C2-C₆ alkenyl that is optionally substituted by at least one of hydroxy and a halogen; and a straight of branched C2-C₆ alkynyl that is optionally substituted by at least one of hydroxy and a halogen;
R11 is selected from a straight or branched C5-C12 alkyl that is optionally substituted by at least one of hydroxy, a halogen, phenyl, and a cycloalkyl; a straight or branched C5-C12 alkenyl that is optionally substituted by at least one of hydroxy, a halogen, phenyl, and a cycloalkyl; and a straight or branched C5-C12 alkynyl that is optionally substituted by at least one of hydroxy, a halogen, phenyl, and a cycloalkyl;
R12 is selected from a straight or branched C1-C6 alkyl;
R13 is selected from H; a straight or branched C1-C6 alkyl; and a straight or branched C1-C6 alkoxy; and
the dotted lines in general formulas II and III depict the bonding pattern of either cyclohexane, phenyl, or a cyclohexene that comprises exactly 1 double bond, which occurs at either A, B, or C.

21. The composition of claim 20, wherein R1 is methyl; ethyl; propyl; butyl; pentyl; hexyl; heptyl; octyl; nonyl; decyl; prop-2-yl; but-2-yl; pent-2-yl; hex-2-yl; hept-2-yl; octan-2-yl; nonan-2-yl; decan-2-yl; 2-methylpropyl; 2-methylbutyl; 2-methylpentyl; 2-methylhexyl; 2-methylheptyl; 2-methyloctyl; 2-methylnonyl; 2-methyldecyl; 2-methylprop-2-yl; 2-methylbut-2-yl; 2-methylpent-2-yl; 2-methylhex-2-yl; 2-methylhept-2-yl; 2-methyloctan-2-yl; 2-methylnonan-2-yl; 2-methyldecan-2-yl; 3-methylbut-2-yl; 3-methylpent-2-yl; 3-methylhex-2-yl; 3-methylhept-2-yl; 3-methyloctan-2-yl; 3-methylnonan-2-yl; 3-methyldecan-2-yl; 2,3-dimethylbut-2-yl; 2,3-dimethylpent-2-yl; 2,3-dimethylhex-2-yl; 2,3-dimethylhept-2-yl; 2,3-dimethyloctan-2-yl; 2,3-dimethylnonan-2-yl; 2,3-dimethyldecan-2-yl; cyclopropyl; 1-methylcyclopropyl; 1-ethylcyclopropyl; 1-propylcyclopropyl; 1-butylcyclopropyl; 1-pentylcyclopropyl; 1-hexylcyclopropyl; 1-heptylcyclopropyl; 1-octylcyclopropyl; 1-nonylcyclopropyl; cyclobutyl; 1-methylcyclobutyl; 1-ethylcyclobutyl; 1-propylcyclobutyl; 1-butylcyclobutyl; 1-pentylcyclobutyl; 1-hexylcyclobutyl; 1-heptylcyclobutyl; 1-octylcyclobutyl; cyclopentyl; 1-methylcyclopentyl; 1-ethylcyclopentyl; 1-propylcyclopentyl; 1-butylcyclopentyl; 1-pentylcyclopentyl; 1-hexylcyclopentyl; 1-heptylcyclopentyl; cyclohexyl; 1-methylcyclohexyl; 1-ethylcyclohexyl; 1-propylcyclohexyl; 1-butylcyclohexyl; 1-pentylcyclohexyl; 1-hexylcyclohexyl; ethenyl; prop-1-enyl; but-1-enyl; pent-1-enyl; hex-1-enyl; hept-1-enyl; octan-1-enyl; nonan-1-enyl; decan-1-enyl; ethynyl; prop-1-ynyl; but-1-ynyl; pent-1-ynyl; hex-1-ynyl; hept-1-ynyl; octan-1-ynyl; nonan-1-ynyl; decan-1-ynyl; 2-phenylethyl; or adamantyl.

22. The composition of claim 20 or 21, wherein R1 is propyl or pentyl.

23. The composition of any one of claims 20-22, wherein R2 is H.

24. The composition of any one of claims 20-23, wherein R3 is H.

25. The composition of any one of claims 20-24, wherein R4 is hydroxy.

26. The composition of any one of claims 20-25, wherein R5 is hydroxy.

27. The composition of any one of claims 20-26, wherein R6 is H; 2-propyl; propen-2-yl; 3-hydroxypropyl; 3-hydroxypropen-2-yl; 4-hydroxy-1-methylbutyl; 4-hydroxy-1-methylbut-2-enyl; 4-hydroxy-1-methylbut-2-ynyl; 1-fluoroethenyl; or 3-fluoropropen-2-yl.

28. The composition of any one of claims 20-27, wherein R6 is propen-2-yl.

29. The composition of any one of claims 20-28, wherein R7 is methyl, hydroxymethyl, fluoromethyl, or oxo.

30. The composition of any one of claims 20-29, wherein R7 is methyl.

31. The composition of any one of claims 20-30, wherein R8 is H.

32. The composition of any one of claims 20-31, wherein R9 is H.

33. The composition of any one of claims 20-32, wherein R10 is H; methyl; 4-methylpent-3-enyl; hydroxymethyl; 3-hydroxypropyl; 3-hydroxyprop-1-enyl; 3-hydroxyprop-1-ynyl; fluoro; or fluoromethyl.

34. The composition of any one of claims 20-33, wherein R10 is methyl.

35. The composition of any one of claims 20-34, wherein R11 is 3-methylbut-2-enyl or 3,7-dimethylocta-2,6-dienyl.

36. The composition of any one of claims 20-35, wherein R12 is methyl.

37. The composition of any one of claims 20-36, wherein R13 is H, methoxy, ethoxy, or 2-propoxy.

38. The composition of any one of claims 20-37, wherein the cannabinoid has a general formula II or III; the dotted lines in general formulas II and III depict the bonding pattern of a cyclohexene; and the cyclohexene comprises exactly 1 double bond, which occurs at either A, B, or C.

39. The composition of any one of claims 20-37, wherein the cannabinoid has a general formula II or III; and the dotted lines in general formulas II and III depict the bonding pattern of phenyl.

40. The composition of any one of claims 1-20, wherein the cannabinoid is selected from cannabidiol; cannabidivarin; cannabidiorcol; cannabidiphorol; tetrahydrocannabinol; tetrahydrocannabivarin; tetrahydrocannabiorcol; tetrahydrocannabiphorol; cannabinol; cannabivarin; cannabigerol; cannabigerovarin; cannabichromene; cannabichromevarin; perrottetinene; delta8-tetrahydrocannabinol; delta8-tetrahydrocannabivarin; 11-hydroxy-tetrahydrocannabinol; 11-hydroxy-tetrahydrocannabivarin; cannabidiol-dimethylheptyl; cannabicyclohexanol; canbisol; nabilone; dexanabinol; parahexyl; dimethylheptylpyran; pirnabine; 11-fluoro-tetrahydrocannabinol; 11-fluoro-tetrahydrocannabivarin; 4′-fluorocannabidiol; HU-331; HU-336; and HU-345.

41. The composition of any one of claims 1-20, wherein the cannabinoid is cannabidiol.

42. The composition of any one of claims 1-20, wherein the cannabinoid is cannabidivarin.

43. The composition of any one of claims 1-20, wherein the cannabinoid is tetrahydrocannabinol.

44. The composition of any one of claims 1-20, wherein the cannabinoid is tetrahydrocannabivarin.

45. The composition of any one of claims 1-20, wherein the cannabinoid is cannabigerol.

46. The composition of any one of claims 1-20, wherein the cannabinoid is cannabigerovarin.

47. The composition of any one of claims 1-46, comprising the cannabinoid at a concentration of at least 10 grams per liter and no greater than 100 grams per liter.

48. The composition of any one of claims 1-46, comprising the cannabinoid at a concentration of at least 1 gram per liter and no greater than 10 grams per liter.

49. The composition of any one of claims 1-46, comprising the cannabinoid at a concentration of at least 100 milligrams per liter and no greater than 1 gram per liter.

50. The composition of any one of claims 1-46, comprising the cannabinoid at a concentration of at least 10 milligrams per liter and no greater than 100 milligrams per liter.

51. The composition of any one of claims 1-46, comprising the cannabinoid at a concentration of at least 1 milligram per liter and no greater than 10 milligrams per liter.

52. The composition of any one of claims 1-51, wherein the composition is formulated for oral administration to a human or an animal.

53. The composition of any one of claims 1-51, wherein the composition is formulated for topical administration to a human or an animal.

54. A medicament, comprising a composition according to any one of claims 1-53.

55. A method to administer a composition to a subject, comprising orally administering a composition according to any one of claims 1-52 to the subject, wherein the subject is a human or an animal.

56. A method to administer a composition to a subject, comprising topically administering a composition according to any one of claims 1-51 and 53 to the subject, wherein the subject is a human or an animal.
